(19) United States
(12) Patent Application Publication
(10) Pub. No.: US 2026/0262964 A1
(43) Pub. Date: Sep. 10, 2026

(54) REMEDIATING SONOGRAPHER MUSCULOSKELETAL STRAIN

(71) Applicant: KONINKLIJKE PHILIPS N.V., EINDHOVEN (NL)

(72) Inventors: Antonio Luigi Perrone, Eindhoven (NL); Faik Can Meral, Mansfield, MA (US); Nicolas Yanez Moreno, Cambridge, MA (US); William Tao Shi, Wakefield, MA (US); Sven Peter Prevrhal, Hamburg (DE); Khaled Salem Abdalleh Younis, Cambridge, MA (US)

(21) Appl. No.: 19/163,430

(22) PCT Filed: Mar. 1, 2024
(86) PCT No.: PCT/EP2024/055352

§ 371 Date: Sep. 9, 2025

Related U.S. Application Data
(60) Provisional application No. 63/451,010, filed on Mar. 9, 2023.

(51) Int. Cl. A61B 5/11; A61B 8/00

(52) U.S. Cl. CPC A61B 5/1116; A61B 8/4254; A61B 8/485; A61B 2503/20

(57) ABSTRACT

A method for monitoring sonographer fatigue, includes obtaining, at a first computer, an estimated posture of a sonographer; obtaining, at the first computer based on data from a sensor (435), an estimated level of repetitive movements by the sonographer; obtaining, at the first computer, an estimated level of pressure applied by the sonographer to a transducer; and determining a level of cumulative physical strain on the sonographer based on the estimated posture, estimated level of repetitive movements, and estimated level of pressure applied by the sonographer.

### BACKGROUND

Medical imaging practices have evolved towards limiting ionizing radiation to reduce the effects of the ionizing radiation on patients. As a result, the role of non-ionizing solutions such as ultrasound in medical imaging has increased. One downside of the increasing use of ultrasound is a steep increase in workload on sonographers and ultrasound systems. In turn, efforts are being made to prevent work-related health effects that result from excessive workload.

Musculoskeletal complaints are one of the primary types of work-related complaints made by sonographers. Work-related musculoskeletal disorders (WRMSD) are a common cause of pain among sonographers, with research suggesting that between 80-90.5% of sonographers may be in pain at least sometimes when performing ultrasound scanning. Work-related musculoskeletal disorders can also lead to sickness, absence, surgical procedures and/or long-term disability. Common causes of work-related musculoskeletal disorders in sonographers include poor and/or static posture, posture changes due to exam variability, repetitive sequences of positions and movements over multiple exams, transducer grip pressure and the use of force, psychosocial factors, and workload management issues. Common symptoms of work-related musculoskeletal disorders include aches and pains, stiffness in the joint, pins and needles sensation, tingling and/or burning sensation. Some sonographers will see evidence of an injury, with physical signs of swelling and/or warmth in the region. Initially, pain may be transient and then improve when not scanning. If no action is taken, injury may progress and pain may become more frequent, eventually leading to a chronic injury which can cause constant pain, in addition to weakness, reduced movement and potentially an inability to carry out every-day tasks.

Ergonomics is the study of human factors affecting workers, with focuses on observing how people interact with the environment they work in and adapting the workplace to the worker, their abilities, and limitations. For sonographers, ergonomics involves assessing the working practices and positions adopted during ultrasound scanning and determining ways to reduce risk of injury for each operator and each type of examination.

To follow the ergonomics approach, a relatively large number of sensors and possibly a camera would be used to sense a current setup for a sonographer and allow for the acquisition of the exact sonographer posture. An implementation using a relatively large number of sensors and a camera can be relatively expensive, complex to operate, and potentially impossible in some contexts due to privacy regulations related to the presence of the patient.

### SUMMARY

According to an aspect of the present disclosure, a method for monitoring sonographer fatigue, includes obtaining, at a first computer, an estimated posture of a sonographer; obtaining, at the first computer based on data from a sensor, an estimated level of repetitive movements by the sonographer; obtaining, at the first computer, an estimated level of pressure applied by the sonographer to a transducer; and determining a level of cumulative physical strain on the sonographer based on the estimated posture, estimated level of repetitive movements, and estimated level of pressure applied by the sonographer.

According to another aspect of the present disclosure, a tangible non-transitory computer-readable storage medium stores a computer program. The computer program, when executed by a processor, causes a system to: obtain an estimated posture of a sonographer; obtain, based on data from a sensor, an estimated level of repetitive movements by the sonographer; obtain an estimated level of pressure applied by the sonographer to a transducer; and determine a level of cumulative physical strain on the sonographer based on the estimated posture, estimated level of repetitive movements, and estimated level of pressure applied by the sonographer.

According to another aspect of the present disclosure, a system includes a memory that stores instructions; and a processor that executes the instructions. When executed by the processor, the instructions cause the system to: obtain an estimated posture of a sonographer; obtain, based on data from a sensor, an estimated level of repetitive movements by the sonographer; obtain an estimated level of pressure applied by the sonographer to a transducer; and determine a level of cumulative physical strain on the sonographer based on the estimated posture, estimated level of repetitive movements, and estimated level of pressure applied by the sonographer.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates a system for remediating sonographer musculoskeletal strain, in accordance with a representative embodiment.

FIG. 1B illustrates another system for remediating sonographer musculoskeletal strain, in accordance with a representative embodiment.

FIG. 1C illustrates another system for remediating sonographer musculoskeletal strain, in accordance with a representative embodiment.

FIG. 2 illustrates a method for remediating sonographer musculoskeletal strain, in accordance with a representative embodiment.

FIG. 3 illustrates another method for remediating sonographer musculoskeletal strain, in accordance with a representative embodiment.

FIG. 4 illustrates another system for remediating sonographer musculoskeletal strain, in accordance with a representative embodiment.

FIG. 5 illustrates a user interface for remediating sonographer musculoskeletal strain, in accordance with a representative embodiment.

FIG. 6 illustrates another system for remediating sonographer musculoskeletal strain, in accordance with a representative embodiment.

FIG. 7 illustrates an ultrasonic probe used in a system for remediating sonographer musculoskeletal strain, in accordance with a representative embodiment.

FIG. 8 illustrates a computer system, on which a method for remediating sonographer musculoskeletal strain is implemented, in accordance with another representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, variable characteristics of an ultrasound system and sonographer activity may be leveraged to determine cumulative musculoskeletal strain of the sonographer.

Sonographer postures may be derived from each type of ultrasound exam performed by the sonographer. Repetitive movements by the sonographer may be derived from movements sensed using a sensor on an ultrasound cord of the ultrasound system. Pressure placed on an ultrasound probe may be derived from elastography. Elastography is an ultrasound technique in which motion induced in tissue is detected by ultrasound reflections to obtain a measure of tissue stiffness underlying the ultrasound probe. The various types of data may be used to estimate and monitor cumulative musculoskeletal strain. Changes of posture, breaks, or other types of feasible remediation measures may be suggested when excessive cumulative musculoskeletal strain is forecasted according to recommendations in guidelines for each category. Sonography procedures may therefore be monitored to detect strain and fatigue signs such as longer times, repetitions and errors, as well as to recommend feasible remediation measures when appropriate. As a result, the overall risk of musculoskeletal injury may be lowered.

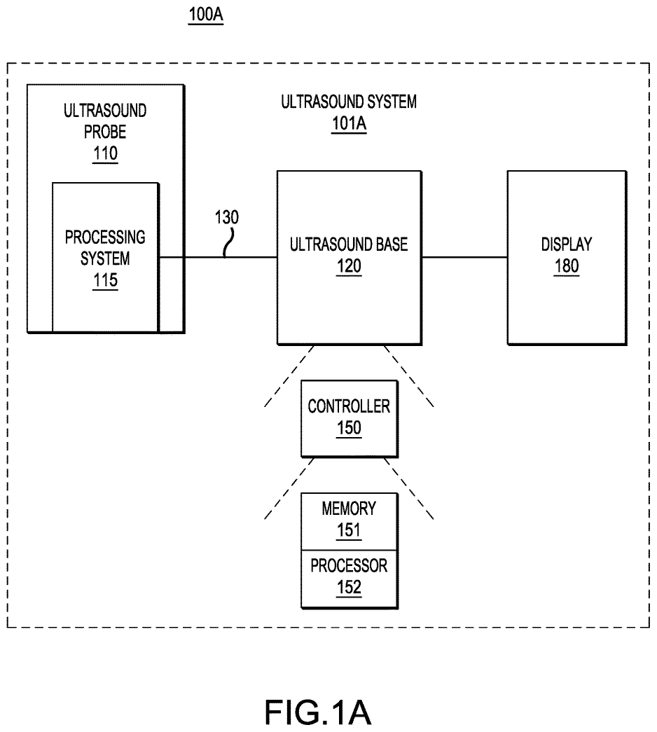

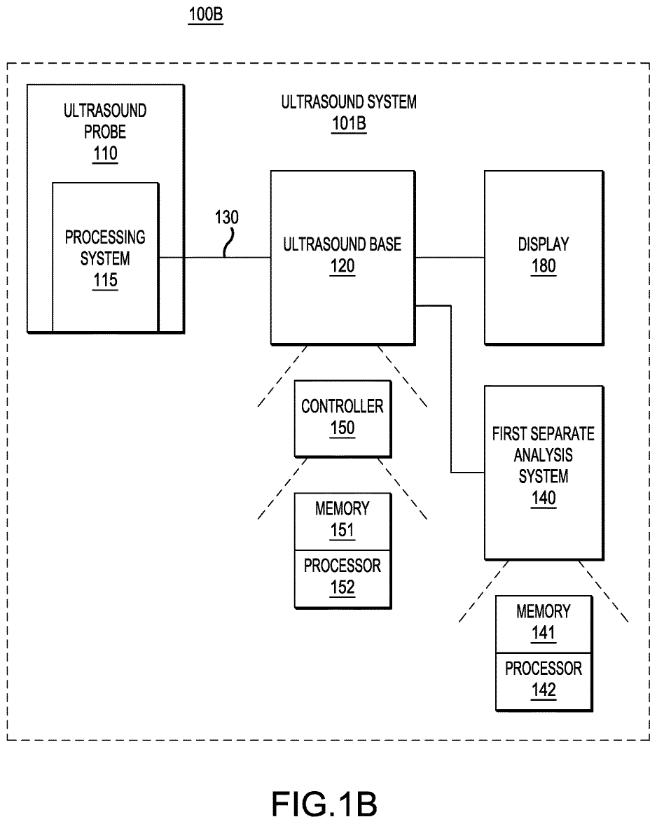

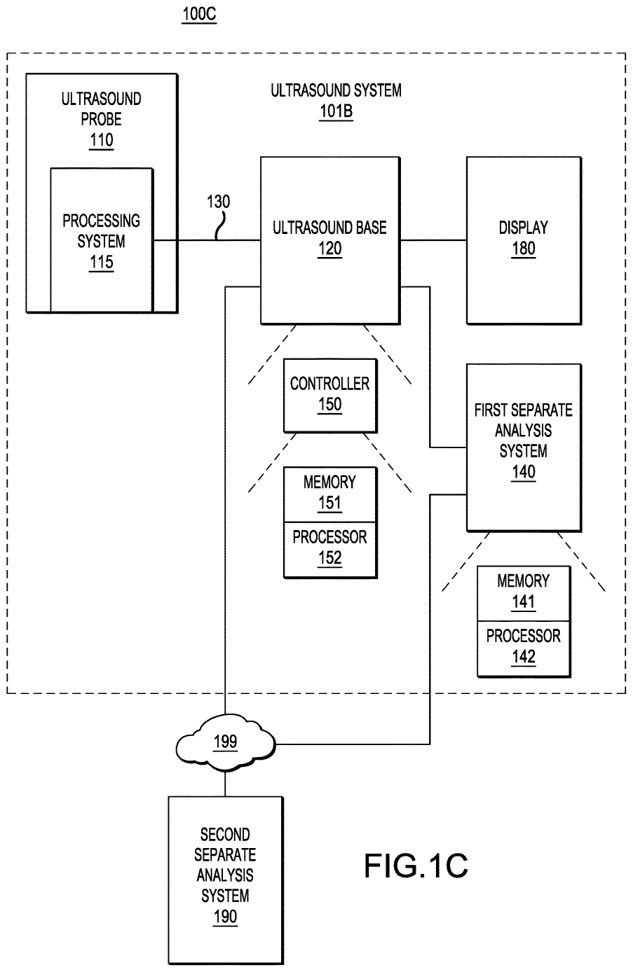

FIG. 1A illustrates a system 100A for remediating sonographer musculoskeletal strain, in accordance with a representative embodiment.

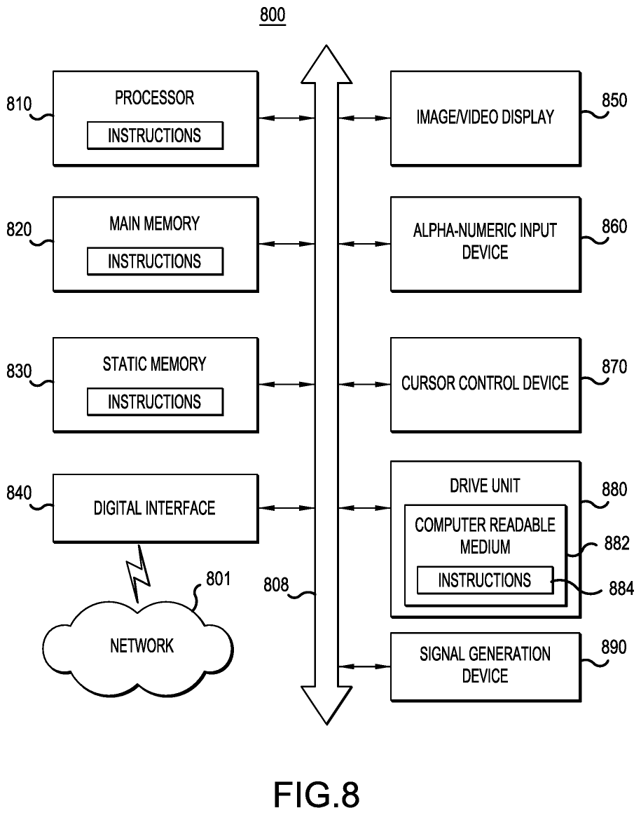

The system 100A in FIG. 1A is a system for remediating sonographer musculoskeletal strain and includes components that may be provided together or that may be distributed. The system 100A includes an ultrasound probe 110, an ultrasound base 120, a cord 130 and a display 180. The ultrasound probe 110 includes a processing system 115. The ultrasound base 120 includes a controller 150. The controller 150 includes at least a memory 151 that stores instructions and a processor 152 that executes the instructions. A computer that can be used to implement the ultrasound base 120 is depicted in FIG. 8, though an ultrasound base 120 may include more or fewer elements than depicted in FIG. 8.

Although not shown in FIG. 1A, one or more separate analysis system or separate analysis systems may be provided with the ultrasound system 101 A and/or remotely. For example, a first separate analysis system may be provided with the ultrasound system 101A to implement elastography as described herein, and a second separate analysis system may be provided as a cloud-based system to determine cumulative musculoskeletal strain as described herein. The first separate analysis system may perform elastography using ultrasound images obtained by the ultrasound probe 110. The second separate analysis system may obtain an estimated posture of the sonographer, an estimated level of repetitive movements by the sonographer, and an estimated level of pressure applied on the ultrasound probe 110 by the sonographer. The second separate analysis system may determine a level of cumulative strain on the sonographer based on the obtained information, and may provide such determinations multiple times in a workday for a single sonographer and for multiple sonographers during their workdays. In some embodiments, the first separate analysis system and the second separate analysis system may be combined either locally or remotely as a single separate analysis system.

The ultrasound probe 110 is connected to the ultrasound base 120 via the cord 130. The processing system 115 may comprise an array of transducers and a processing circuit. The array of transducers convert electrical energy into sound waves which bounce off of body tissue, and receive echoes of the sound waves and convert the echoes into electrical energy. The array of transducers may include dozens, hundreds or thousands of individual transducer elements. The ultrasound probe 110 may transmit a beam to produce images and may detect the echoes. The processing system 115 may process ultrasound images captured by the ultrasound probe 110.

The ultrasound base 120 is connected to the ultrasound probe 110 via the cord 130. The ultrasound base 120 may be or otherwise include an ultrasound cart. In addition to the controller 150, the ultrasound base 120 may include buttons as user interfaces. A main user interface of the ultrasound base 120 may be or include buttons. The buttons may correspond to different functions of the ultrasound base 120, and sonographers may be well versed to recognize which buttons to press to control different functions of the ultrasound base 120 and the ultrasound probe 110. To some extent, any action performed for an ultrasound session may be prepared, selected, and optimized by some sequence of button pushes. Performance of ultrasound procedures by a sonographer may rely on the peculiarities of the ultrasound base 120, including reliance of the ultrasound probe 110 on the ultrasound base 120, and a workflow that centers on the ultrasound base 120. Some or all activity by the sonographer related to the ultrasound examination may be reflected by some action on the ultrasound base 120. Relevant actions for an ultrasound examination may be prepared, selected, and optimized by some specific sequence of buttons on the ultrasound base 120. Sequences of pushes of buttons on the ultrasound base 120 may be acquired and stored in log-files maintained by and/or for the ultrasound base 120.

The controller 150 includes at least the memory 151 and the processor 152. The controller 150 may also include other types of interfaces such as ports, disk drives, wireless antennas, or other types of receiver circuitry that connect the controller 150 to other electronic elements. The memory 151 may store instructions executed by the processor 152. The memory 151 may also store log-files as they are created by and for the ultrasound base 120. The log-files may store records of types of ultrasound procedures performed by the sonographer and records of sequences of button pushes performed during each procedure. An example of a log-file includes an ordered arrangement of data labeled for events and inputs provided via the user interfaces. The ordered arrangement of data may include timestamps, input types, types of data such as workflow, and sequences of buttons pushed during a workflow. The sequences of button pushes may also be provided as statistics, such as counts and percentages for specific actions such as freezes, acquisitions, changes of depths, zooming, scanning, erasing, updating and other types of actions taken when specific buttons on the ultrasound base 120 are pushed.

The display 180 may be local to the ultrasound base 120. The display 180 may be connected to the ultrasound base 120 via a local wired interface such as an Ethernet cable or via a local wireless interface such as a Wi-Fi connection. The display 180 may be interfaced with other user input devices by which users can input instructions, including mouses, keyboards, thumbwheels and so on. The display 180 may be a monitor such as a computer monitor, a display on a mobile device, an augmented reality display, a television, an electronic whiteboard, or another screen configured to display electronic imagery. The display 180 may also include one or more input interface(s) such as those noted above that may connect to other elements or components, as well as an interactive touch screen configured to display prompts to users and collect touch input from users.

The processing system 115 and/or the controller 150 may perform some of the operations described herein directly and may implement other operations described herein indirectly. For example, the processing system 115 may directly implement operations for the ultrasound probe 110. The controller 150 may directly control operations such as logical operations performed by the processor 152 executing instructions from the memory 151 based on input received from electronic elements and/or users via the interfaces. The processes implemented by the controller 150 may also include steps not directly performed by the controller 150. For example, the controller 150 may indirectly control operations such as by generating and transmitting content to be displayed on the display 180. Of course, the particular arrangement and relative functionality of controller 150 and processing system 115 may vary within the scope of the present embodiments.

Among the technical features performed by the ultrasound system 101A, the controller 150 may execute software to perform a variety of functions described herein. For example, the controller 150 may execute instructions to acquire and process the log-files stored in the memory 151 for extraction of the relevant data. The relevant data may include, for example, button pushes, timestamps, delta times, acquisition settings, exam type, and if an ultrasound image is acquired. The controller 150 may also execute instructions to estimate the current ultrasound equipment setup, such as whether the sonographer is sitting or standing based on the type of ultrasound procedure, the configuration of the ultrasound base 120, and/or demographic characteristics of the sonographer. Demographic data of a patient may also be taken into account when the data may reflect that the patient demographics may contribute to sonographer musculoskeletal strain. Patient data that may be taken into account may include height or weight, for example. The controller 150 may further execute instructions to estimate the current sonographer posture, such as based on the type of ultrasound procedure being performed as well as which buttons are being pushed by the sonographer during the ultrasound procedure. Posture may be determined based on the log-file data which reflects which buttons are pushed and/or based on input received directly from the sonographer indicating the type of procedure being performed.

The controller 150 may also determine levels of repetitive actions performed by the sonographer. A sensor may be provided on the cord 130. The sensor may be a hardware sensor used to measure distances and movements to determine the repetitions of the movement of the sonographer. The sensor may be used to determine repetition without requiring visual information such as from a camera. The sensor may be accurate to a degree lower than a millimeter, and provides detailed definition and historical data on the repetitive movement of the sonographer. The controller 150 may estimate the current movement repetition level using data from the sensor on the cord 130. The sensor on the cord 130 may be a new type of hardware in the context of the ultrasound system 101A, and may comprise a telescopic draw-wire displacement sensor integrated in the cord 130 using micro deflection pulleys. The telescopic draw-wire displacement sensor may be used determine a displacement of the sensor, and an estimated level of repetitive movements by the sonographer may be estimated based on the determined displacement of the sensor.

Additionally, the controller 150 or a first separate analysis system local to the ultrasound system 101A may perform elastography and then send the elastography results to a second separate analysis system.

The configuration of the ultrasound system 101A in FIG. 1A does not particularly require a camera monitoring the sonographer. While a camera may be used to monitor the sonographer to provide inputs for the analyses described herein, such a camera is not particularly necessary for the ultrasound system 101A. Instead, the log-file data from the ultrasound base 120, other information input by the sonographer, and repetition data derived from the sensor on the cord 130 may be the inputs used for the analyses described herein, even though other types of information can be used as enhancements in some embodiments.

FIG. 1B illustrates another system for remediating sonographer musculoskeletal strain, in accordance with a representative embodiment.

In FIG. 1B, the system includes an ultrasound system 101B. In addition to the elements of the system 100A in FIG. 1A, the ultrasound system 100B also includes a first separate analysis system 140. The first separate analysis system 140 includes a memory 141 and a processor 142. The first separate analysis system 140 is local to the ultrasound base 120, and may be configured to perform elastography as described herein. That is, instructions stored in the memory 141 may be executed by the processor 142 to process ultrasound images received from the ultrasound base 120. The memory 141 may also include one or more structural models for anatomy to use in performing the elastography. The elastography may be performed to determine pressure applied to the ultrasound probe 110 based on analysis of the ultrasound images captured by the ultrasound probe 110. The elastography may also rely on characteristics of each ultrasound exam, such as using the structural models which correspond to the type of each ultrasound exam. The structural models to use in the elastography may be determined based on the log-file data stored in the memory 151 of the controller 150.

FIG. 1C illustrates another system for remediating sonographer musculoskeletal strain, in accordance with a representative embodiment.

In FIG. 1C, the system 100C includes the ultrasound system 101B from FIG. 1B. In addition to the elements of the ultrasound system 101B in FIG. 1B, the system 100C also includes a network 199 and a second separate analysis system 190. The network 199 may comprise a wide-area network such as the Internet. The second separate analysis system 190 may comprise a remote system which is remote from the ultrasound system 101B. For example, the second separate analysis system 190 may comprise a cloud-based system implemented using one more data centers each configured with combinations of servers and large memories paired with the servers. The second separate analysis system 190 may perform determinations of cumulative physical strain for one or more sonographers at one or more facilities. The second separate analysis system 190 may perform such determinations 24 hours a day, 7 days a week and 365 days a year, for example, as a service for sonographers distributed geographically.

The second separate analysis system 190 may be a cloud-based system and may evaluate the pressure applied by the sonographer on the transducer of the ultrasound probe 110. Current sonographer physical strain may be cumulatively determined from the various data collected from monitoring the data from the ultrasound session, including from the ultrasound images. The current sonographer physical strain may be compared against a threshold to evaluate whether alternative standard modalities should be suggested for performing selected types of ultrasound acquisitions, or whether the sonographer should take a break or perform regenerative exercises. As noted above, various demographic data of the sonographer may be taken into account when evaluating cumulative strain.

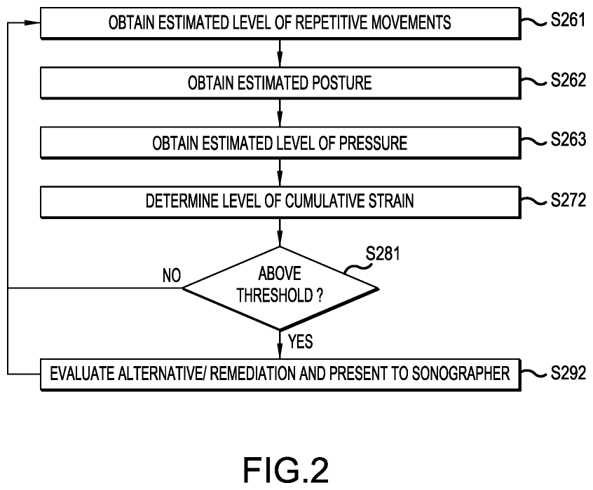

FIG. 2 illustrates a method for remediating sonographer musculoskeletal strain, in accordance with a representative embodiment.

The teachings herein provide solutions for minimizing or preventing musculoskeletal and ergonomic strain during the normal ultrasound operations by a sonographer. The teachings help alleviate musculoskeletal disorders insofar as musculoskeletal disorders are a common cause of pain and sickness absence for sonographers.

The method of FIG. 2 starts at S261 with obtaining an estimated level of repetitive movements of a sonographer. Repetitive movements are a common source of musculoskeletal disorders, and the level of repetitive movements may be performed by monitoring determined displacements of a sensor on the cord 130.

At S262, an estimated posture or postures is obtained. Data used to estimate posture may include data of whether the sonographer is sitting or standing, and whether the sonographer is likely to be stooping or straining. Posture may be estimated from the type of ultrasound procedure being performed and known physical characteristics of the ultrasound base 120 and/or demographic characteristics of the sonographer and/or the patient. Posture may be estimated from log-file data from the ultrasound base 120, such as sequences of buttons on the ultrasound base 120 pushed by the sonographer and relative positions of each button. Posture may be determined based on input directly from the sonographer or based on determinations derived from input directly from the sonographer. The estimation at S262 may be performed without requiring input from any camera monitoring the sonographer for ergonomic information.

At S263, an estimated level of pressure is obtained. Excessive pressure on an ultrasound probe and poor grip on the ultrasound probe are common sources of musculoskeletal disorders. Elastography may be used to estimate the level of pressure at S263. Elastography is used to derive the level of pressure on the ultrasound probe 110 using ultrasound images insofar as pressure applied to an ultrasound probe 110 results in shear waves which are horizontal rather than perpendicular. From the movement derivable from the acquired ultrasound images, the amount of pressure that has been applied to the ultrasound probe 110 may be inferred. The type of examination being conducted may also be used insofar as this type of information reflects where the ultrasound probe 110 is going to be. Using the information from the ultrasound images, the type of examination and the characteristics of the patient body, an estimated level of pressure on the ultrasound probe 110 may be estimated. In some embodiments, the sequences of button pushes reflected in the log-file data may be used to determine a mechanical model of tissue for the examined area, and the determined mechanical model may be used in the elastography performed to derive the level of pressure. In some embodiments, S263 may be performed by the first separate analysis system 140 in FIG. 1B and FIG. 1C. In other embodiments, S263 may be performed by the second separate analysis system 190 in FIG. 1C, such as when the ultrasound images are sent to a remote cloud-based service which performs the elastography.

At S272, a level of cumulative strain is determined. The level of cumulative strain may be determined from estimations at S261, S262 and S263. For example, the cumulative strain may reflect strain from an individual ultrasound session, or from multiple ultrasound sessions involving the same sonographer in a continuous sequence or over a period of time such as a workday. S272 may be performed by the second separate analysis system 190 in FIG. 1C, and may be performed for multiple sonographers and multiple ultrasound systems.

At S281, a determination is made as to whether the level of cumulative strain is above a threshold. S281 may be performed by the second separate analysis system 190 in FIG. 1C, and may be performed for multiple sonographers and multiple ultrasound systems.

If the level of cumulative strain is above the threshold (S281=Yes), at S292 alternative potentially feasible remediation measures are evaluated and presented to the sonographer. The evaluation at S292 may be performed by the second separate analysis system 190 in FIG. 1C, and the presentation at S292 may be performed using the display 180. The estimations and determinations in FIG. 2 may be automated estimations of the cumulative sonographer musculoskeletal strain, and may be performed throughout a sonographer's workday. By passively monitoring the sonographer, alternative feasible remediation measures may be offered when appropriate. Alternative remediation measures may include preventive measures such as alternative procedural standard modalities, breaks or recovery exercises.

If the level of cumulative strain is not above the threshold (S281=No), the method of FIG. 2 returns to S261.

Other types of factors may also be taken into account in the method of FIG. 2. For example, stress or workload resulting from workload management issues may be taken into account. An example of a stress or workload factor may be the length of a workday such as a workday being extended from 8 hours to 12 hours. Another example of a stress or workload factor may be the number of consecutive days worked such as a sonographer performing sonography for 4 or more hours for each of 6 or more consecutive days. As another example, stress or workload may result from psychosocial factors such as limited support for a sonographer.

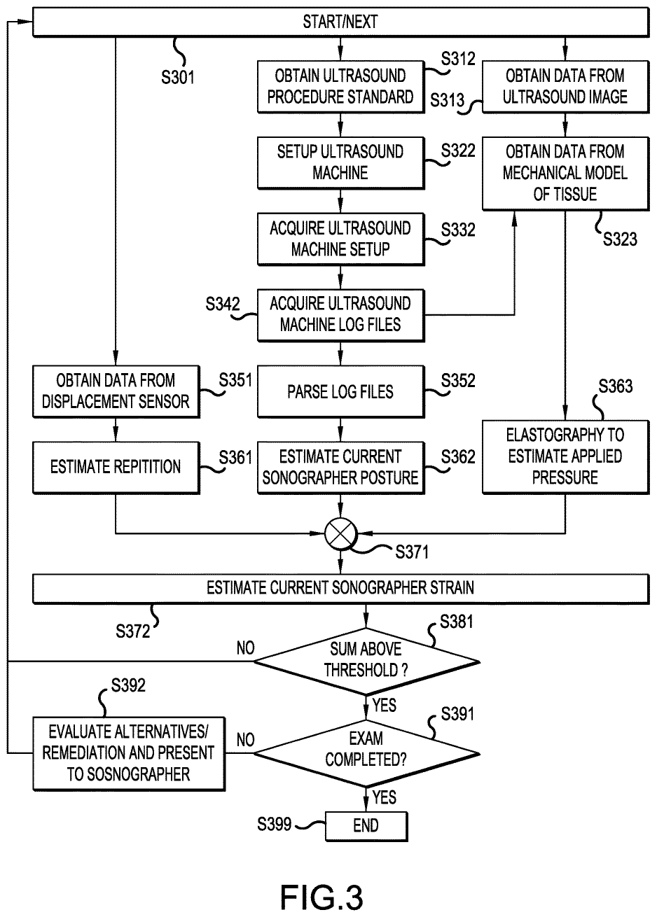

FIG. 3 illustrates another method for remediating sonographer musculoskeletal strain, in accordance with a representative embodiment.

The method of FIG. 3 may be performed largely or entirely using software modules executed by the controller 150, the first separate analysis system 140 and/or the second separate analysis system 190.

The method of FIG. 3 starts at S301. The method of FIG. 3 includes steps primarily shown in three columns on the left, right and middle. The left column includes steps relating directly to the use of a displacement sensor, such as the sensor attached to the cord 130. The middle column includes steps relating directly to the use of an ultrasound machine, such as the ultrasound base 120 and the ultrasound probe 110. The right column includes steps relating directly to the use of an analysis system that analyzes ultrasound images, such as the first separate analysis system 140 or the second separate analysis system 190.

The middle column of steps relating directly to the use of an ultrasound machine is described first. At S312, an ultrasound procedure standard is obtained. S312 relates to features performed directly in relation to an ultrasound machine such as the ultrasound base 120. The ultrasound procedure standard obtained at S312 may be selected from a set of multiple localized standard procedures for all the types of ultrasound exams the sonographer can perform. Such ultrasound procedure standards are detailed guidelines that include exact specifications of the type of locations and movements that the ultrasound probe 110 must follow for a correct examination. Standard practice is that in case there are not explicit national guidelines on the subject they are borrowed from other countries and adjusted to the local environment. The evaluations may be adjusted according to the variations detected by the analysis of log-files and movements repetition.

At S322, the ultrasound machine is set up. The ultrasound machine set up at S332 may comprise the ultrasound probe 110 and the ultrasound base 120.

At S332, the ultrasound machine setup is acquired. The ultrasound machine setup acquired at S332 may be acquired by the second separate analysis system 190.

At S342, ultrasound machine log files are acquired. The log files contain log-file data that can be used to estimate the estimated posture of the sonographer and that can be used to determine the mechanical models for elastography. The log-file data may include one or more sequence(s) of buttons on the ultrasound base 120 pushed by the sonographer.

At S352, the ultrasound machine log file-data is parsed. A software module may parse the log-file data written in real time during each ultrasound examination. The software module may extract the pushed buttons, timestamps associated with each button push, and any other specific information which may be used to estimate sonographer posture.

At S362, a current sonographer posture is estimated. All the above data collected at S312, S332 and S342 may be used to form the input for the estimation of current sonographer posture. From the standard sequence of events depicted in the guidelines obtained at S312, the associated posture may be known. The timestamps between the button pushes obtained from the log-file data at S342 allow estimation of the length of the posture and even deviations from the guidelines.

The right column of steps relating directly to an analysis system are described next. At S313, data is obtained from an ultrasound image. The data may be obtained by analyzing ultrasound images obtained from the ultrasound base 120. For most embodiments herein, data from ultrasound images is acquired from the real-time feed from the ultrasound probe 110.

At S323, data is obtained from a mechanical model of tissue. The data may be obtained at S323 from the memory 141. The data from ultrasound images acquired at S313 is integrated with a standard mechanical model of tissue around the examined area obtained at S323. The standard mechanical model of tissue to use may be determined based on log-file data acquired at S342 as well as from the data from ultrasound images obtained at S313.

At S363, elastography may be performed to estimate applied pressure. Elastography is used to determine the physical strain (or force or pressure) applied to the ultrasound probe 110 during the ultrasound exam. The data obtained at S313 and S332 may form the input to a trained artificial intelligence model, integrated with the physical/mechanical model obtained at S323, and trained to estimate the applied probe pressure from the deformations shown in the ultrasound images. The deformations include vertical/displacement and longitudinal/shear as shown in the ultrasound images.

Next, the left column of steps relating directly to the ultrasound base 120 is described. At S351, data is obtained from a displacement sensor on the cord 130. The data obtained at S351 is collected over time based on motion of the displacement sensor on the cord 130.

At S361, repetition is estimated based on the data of determined displacements obtained based on the displacement sensor. The data collected at S351 is used at S361.

Based on the steps in the left column and the middle column, a clear picture of the sonographer posture and repetition of movements between button pushes is known. Based on the steps in the right column, the additional inputs obtained from elastography are known. The collected data may be provided to the second separate analysis system 190 during or after each ultrasound examination performed by a sonographer.

At S371, inputs from S361, S362 and S363 are obtained to estimate the current sonographer strain. The inputs may be summed or otherwise combined to reflect one or more values that can be compared to a threshold at S381. The current cumulative musculoskeletal strain of the sonographer is estimated at S372. At S372, current sonographer musculoskeletal strain is estimated based on the inputs of the repetition estimated at S361, the current sonographer posture estimated at S362, and the elastography performed at S363. Additionally, previous value or values obtained from previous sonography sessions by the sonographer.

S372 is based on all the information acquired and estimated from the steps performed on the left column, the middle column and the right column in FIG. 3. The estimation at S372 may be performed by an artificial intelligence model integrating the three main strain variables of posture, movement repetition and probe pressure. The artificial intelligence model may be implemented in a cloud-based system and may adjust the estimation from the standard procedures taking into account deviations which are evident from the analysis of the workflow as registered in the log-files from the ultrasound base 120. The artificial intelligence model may also take as input to the amount of time spent by the sonographer in each phase, the number of repetitions, and other types of relevant input. The artificial intelligence model may evaluate the various input data to filter out when the sonographer is speaking with a patient or pausing, such as when no movements are detected on the hardware sensor on the cord 130 and when no ultrasound images are acquired.

At S381, a determination is made as to whether the total of the cumulative strain is above a threshold. Once the estimation at S371 is completed, the determination at S381 is performed to decide if the current level of strain is higher than a mandated given amount such as a threshold.

If the sum is not above the threshold (S381=No), the process returns to S301.

If the sum is above the threshold (S381=Yes), a determination is next made at S391 as to whether the exam is completed. If the exam is completed (S391=Yes), the method of FIG. 3 ends at S399.

If the exam is not completed (S391=No), but the sum is above the threshold (S381=Yes), alternative potentially feasible remediation measures are evaluated and presented to the sonographer at S392, and then the process returns to S301. Alternative feasible remediation measures may be provided by message audio or displays aimed to suggest alternatives such as alternative movements to perform the exam and/or suggestions to the sonographer to take a break. Alternatives feasible remediation measures may also include suggestions of some restorative exercise for the most affected parts such as hand, shoulder or neck as defined by the strain analysis.

An algorithm run locally in the ultrasound base 120 may run until the end of each ultrasound examination, and provide data and/or ultrasound images to the first separate analysis system 140 and/or to the second separate analysis system 190.

Although FIG. 3 shows the process ending at S399 when the current ultrasound exam is complete, remediation may still be suggested to the sonographer before ending the process. For example, if the cumulative sonographer strain is above the threshold by more than a predetermined amount, the sonographer may be warned to take a break before the next ultrasound exam. In some embodiments, the second separate analysis system 190 may notify an administrator when cumulative strain on a sonographer is above a threshold by a predetermined amount, so that the administrator may intervene when appropriate.

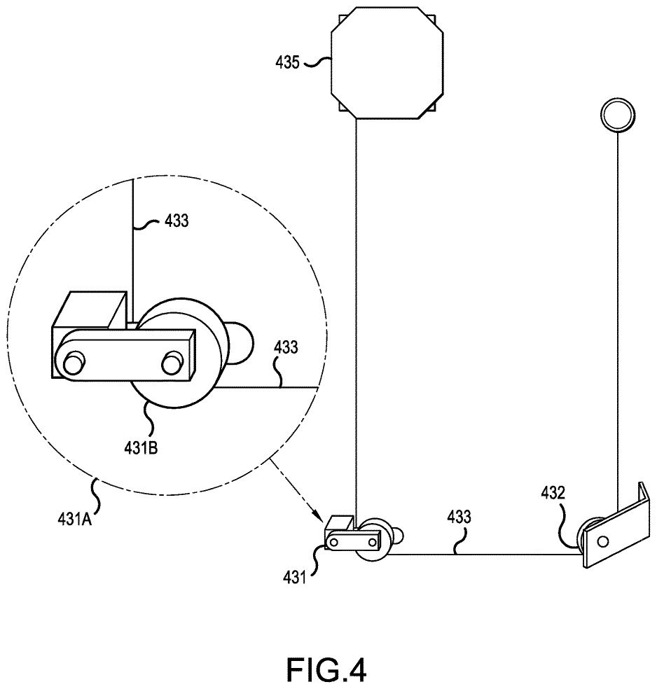

FIG. 4 illustrates another system for remediating sonographer musculoskeletal strain, in accordance with a representative embodiment.

FIG. 4 shows an example of a telescopic draw-wire displacement sensor using deflection pulleys. The system shown in FIG. 4 includes a first pulley 431, a second pulley 432, a cord 433, and a sensor 435. The cord 433 may comprise a portion of the sensor 435 and is designated by a circular feature representing a distal end of a cord. The sensor 435 is a displacement sensor and may comprise another pulley with a wire wrapped around, such that an end of the wire is attached to the pulley. The distal end of the cord 433 is away from a casing of a sensor body of the sensor 435 comprising the pulley with a proximal end of the cord 433 wrapped around. The pulley of the sensor 435 may be fixed to the ultrasound base 120. The first pulley 431 includes an outer protective surface 431A and a spool 431B, and the cord 433 of the sensor 435 is wrapped partially around the spool 431B. The second pulley 432 may include a configuration similar to the first pulley 431. The casing of the sensor body of the sensor 435 may be attached directly or indirectly to the ultrasound base 120. For example, the sensor 435 may be attached to the ultrasound base 120 close to where the probe cable is attached, though this is not specifically required. The distal end of the sensor 435 designated by the circular feature of the cord 433 may be attached directly to the ultrasound probe 110 or near the probe cable close to the ultrasound probe 110. The sensor 435 measures how many times the wire reels or unreels around its pulley, including partial reels or unreels. Accordingly, the casing of the sensor 435 is fixed to the ultrasound base 120, and the internal movement of the wire by reeling or unreeling is sensed. As the wire of the sensor 435 is displaced based on movement of the cord 433, movement of the wire of the sensor 435 may be sensed and recorded as data.

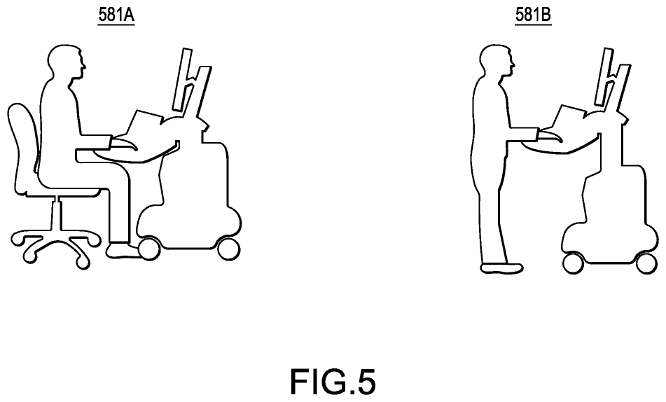

FIG. 5 illustrates a user interface for remediating sonographer musculoskeletal strain, in accordance with a representative embodiment.

A software module executed by the controller 150 may detect the setup of an ultrasound system including the ultrasound base 120. The detection may involve determining whether the sonographer is standing or sitting. The setup may be read from the log-files stored in the memory 151, deduced by applying artificial intelligence to data from the ultrasound base 120, or even obtained directly from the sonographer via input to the ultrasound base 120. Alternatively, sensors placed in the ultrasound system display and keyboard positional adjustment arms may provide data to the controller 150 that indicates the estimated posture of the sonographer.

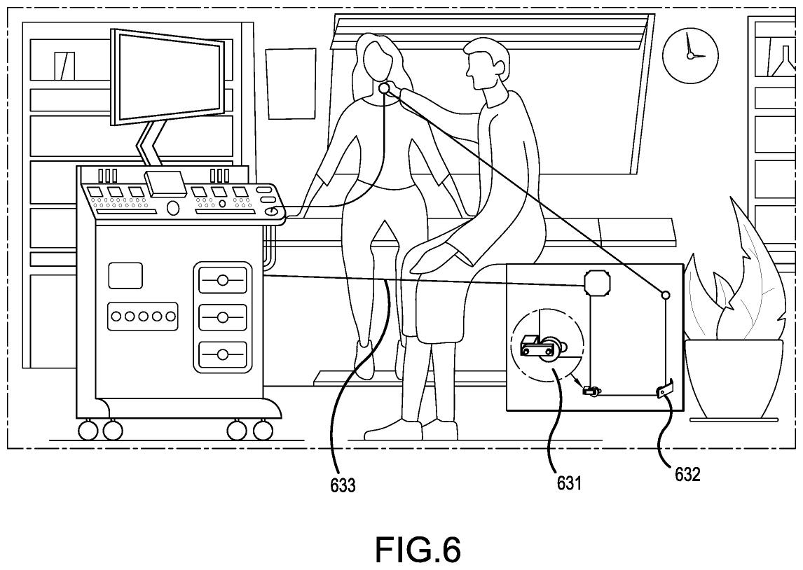

FIG. 6 illustrates another system for remediating sonographer musculoskeletal strain, in accordance with a representative embodiment.

The system in FIG. 6 includes a first pulley 631, a second pulley 632 and a cord 633. As shown, the pulleys are fixed deflection pulleys along the cord 633 from the ultrasound probe to the ultrasound base and are used to follow the repetitions of the ultrasound probe movements by its biunivocal association with the measured displacements of the draw-wire sensor. That is, the elongation of the cord 633 is sensed by the draw-wire sensor, and this is used to determine the level of repetitions by the sonographer. An alternative embodiment of this arrangement (not shown) integrates the pulleys and cord into the probe cable itself. The pulleys and cord are attached at suitable locations along the probe cable for compactness and ease of use. The level of repetition is used to better specify the strain between button pushes.

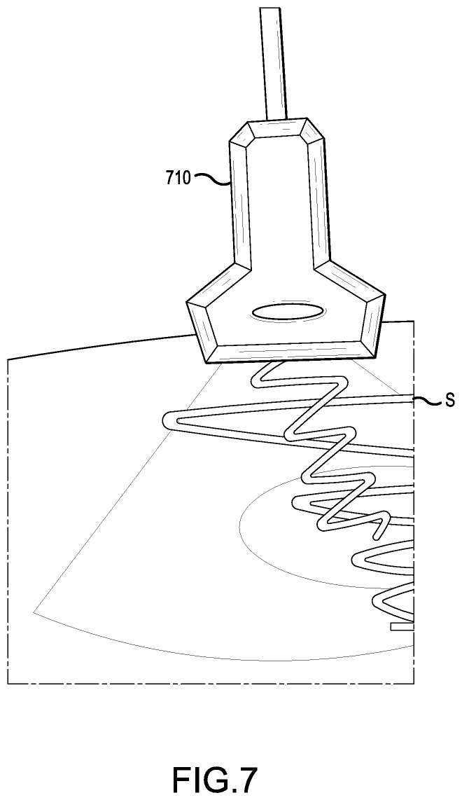

FIG. 7 illustrates an ultrasonic probe used in a system for remediating sonographer musculoskeletal strain, in accordance with a representative embodiment.

FIG. 7 shows the elastography principle for ultrasound using pressure and shear waves caused by the ultrasound probe. One or more shear wave S is/are labelled in FIG. 7. The ultrasound probe 710 shown in FIG. 7 may correspond to the ultrasound probe 110 in FIG. 1A. Elastography is used to estimate the pressure applied by the sonographer on the ultrasound probe 710 without requiring any additional sensor such as a monitoring camera. The elastography may be performed using a software module executed by the controller 150 or by the first separate analysis system 140 or the second separate analysis system 190.

FIG. 8 illustrates a computer system, on which a method for remediating sonographer musculoskeletal strain is implemented, in accordance with another representative embodiment.

Referring to FIG. 8, the computer system 800 includes a set of software instructions that can be executed to cause the computer system 800 to perform any of the methods or computer-based functions disclosed herein. The computer system 800 may operate as a standalone device or may be connected, for example, using a network 801, to other computer systems or peripheral devices. In embodiments, a computer system 800 performs logical processing based on digital signals received via an analog-to-digital converter.

In a networked deployment, the computer system 800 operates in the capacity of a server or as a client user computer in a server-client user network environment, or as a peer computer system in a peer-to-peer (or distributed) network environment. The computer system 800 can also be implemented as or incorporated into various devices, such as the ultrasound base 120, a workstation that includes a controller, the first separate analysis system 140, the second separate analysis system 190, a stationary computer, a mobile computer, a personal computer (PC), a laptop computer, a tablet computer, or any other machine capable of executing a set of software instructions (sequential or otherwise) that specify actions to be taken by that machine. The computer system 800 can be incorporated as or in a device that in turn is in an integrated system that includes additional devices. In an embodiment, the computer system 800 can be implemented using electronic devices that provide voice, video or data communication. Further, while the computer system 800 is illustrated in the singular, the term “system” shall also be taken to include any collection of systems or sub-systems that individually or jointly execute a set, or multiple sets, of software instructions to perform one or more computer functions.

As illustrated in FIG. 8, the computer system 800 includes a processor 810. The processor 810 may be considered a representative example of a processor of a controller and executes instructions to implement some or all aspects of methods and processes described herein. The processor 810 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 810 is an article of manufacture and/or a machine component. The processor 810 is configured to execute software instructions to perform functions as described in the various embodiments herein. The processor 810 may be a general-purpose processor or may be part of an application specific integrated circuit (ASIC). The processor 810 may also be a microprocessor, a microcomputer, a processor chip, a controller, a microcontroller, a digital signal processor (DSP), a state machine, or a programmable logic device. The processor 810 may also be a logical circuit, including a programmable gate array (PGA), such as a field programmable gate array (FPGA), or another type of circuit that includes discrete gate and/or transistor logic. The processor 810 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 800 may further include a main memory 820 and a static memory 830, where memories in the computer system 800 communicate with each other and the processor 810 via a bus 808. Either or both of the main memory 820 and the static memory 830 may be considered representative examples of a memory of a controller, and store instructions used to implement some, or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 820 and the static memory 830 are articles of manufacture and/or machine components. The main memory 820 and the static memory 830 are computer-readable mediums from which data and executable software instructions can be read by a computer (e.g., the processor 810). Each of the main memory 820 and the static memory 830 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 800 further includes a video display unit 850, such as a liquid crystal display (LCD), an organic light emitting diode (OLED), a flat panel display, a solid-state display, or a cathode ray tube (CRT), for example. Additionally, the computer system 800 includes an input device 860, such as a keyboard/virtual keyboard or touch-sensitive input screen or speech input with speech recognition, and a cursor control device 870, such as a mouse or touch-sensitive input screen or pad. The computer system 800 also optionally includes a disk drive unit 880, a signal generation device 890, such as a speaker or remote control, and/or a network interface device 840.

In an embodiment, as depicted in FIG. 8, the disk drive unit 880 includes a computer-readable medium 882 in which one or more sets of software instructions 884 (software) are embedded. The sets of software instructions 884 are read from the computer-readable medium 882 to be executed by the processor 810. Further, the software instructions 884, when executed by the processor 810, perform one or more steps of the methods and processes as described herein. In an embodiment, the software instructions 884 reside all or in part within the main memory 820, the static memory 830 and/or the processor 810 during execution by the computer system 800. Further, the computer-readable medium 882 may include software instructions 884 or receive and execute software instructions 884 responsive to a propagated signal, so that a device connected to a network 801 communicates voice, video or data over the network 801. The software instructions 884 may be transmitted or received over the network 801 via the network interface device 840.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, variable characteristics of an ultrasound and sonographer activity may be leveraged to determine cumulative musculoskeletal strain. Log-file data from ultrasound machines may be used to identify procedures and how such procedures evolve over time. Other types of information such as patient biometric data may also be used to predict sonographer fatigue. Various types of data may be used to estimate and monitor sonographer posture according to standard positioning for each type of ultrasound procedure, and evaluate cumulative musculoskeletal strain. Changes of posture, breaks, or other types of feasible remediation measures may be suggested when excessive cumulative strain is forecasted according to recommendations in guidelines for each category. Sonography procedures may therefore be monitored to detect strain, and fatigue signs such as longer times, repetitions and errors, recommend remediation measures when appropriate, and lower the overall risk of musculoskeletal injury.

Although remediating sonographer musculoskeletal strain has been described with reference to several exemplary embodiments, it is understood that the words that have been used are words of description and illustration, rather than words of limitation. Changes may be made within the purview of the appended claims, as presently stated and as amended, without departing from the scope and spirit of remediating sonographer musculoskeletal strain in its aspects. For example, the teachings herein are not limited to configurations with ultrasound carts; instead, the teachings herein may be applicable to other types of ultrasound configurations including ultraportable configurations. Although remediating sonographer musculoskeletal strain has been described with reference to particular means, materials and embodiments, remediating sonographer musculoskeletal strain is not intended to be limited to the particulars disclosed; rather remediating sonographer musculoskeletal strain extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept.

Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method for monitoring sonographer fatigue, comprising:
obtaining, at a first computer, an estimated posture of a sonographer;
obtaining, at the first computer based on data from a sensor an estimated level of repetitive movements by the sonographer;
obtaining, at the first computer, an estimated level of pressure applied by the sonographer to a transducer; and
determining a level of cumulative physical strain on the sonographer based on the estimated posture, estimated level of repetitive movements, and estimated level of pressure applied by the sonographer.

2. The method of claim 1, further comprising:
obtaining log-file data from an ultrasound cart; and
estimating the estimated posture of the sonographer at the first computer based on the log-file data.

3. The method of claim 2, wherein the log-file data comprises at least one sequence of buttons on the ultrasound cart pushed by the sonographer.

4. The method of claim 1, further comprising:
determining whether to propose one or more remediation measures to relieve the cumulative physical strain on the sonographer; and
determining one or more feasible remediation measures based on determining to propose one or more remediation measures to relieve the cumulative physical strain.

5. The method of claim 1, further comprising:
determining a displacement of the sensor, wherein the sensor is attached to an ultrasound probe directly or integrated in or on a cord of the ultrasound probe and
estimating the estimated level of repetitive movements by the sonographer based on the determined displacement of the sensor.

6. The method of claim 1, further comprising:
determining a type of each of one or more procedures performed by the sonographer;
determining a setup of an ultrasound system used by the sonographer; and
determining the level of cumulative physical strain on the sonographer based further on the type of each of the one or more procedures performed by the sonographer and the setup of the ultrasound system used by the sonographer.

7. The method of claim 1, further comprising:
obtaining ultrasound images taken by an ultrasound system;
applying elastography to the ultrasound images taken by the ultrasound system; and
estimating the estimated level of pressure applied by the sonographer to the transducer using elastography based on the elastography applied to the ultrasound images.

8. The method of claim 7, wherein the estimated level of pressure is estimated at a second computer and sent from the first computer to the second computer.

9. A tangible non-transitory computer-readable storage medium that stores a computer program, wherein the computer program, when executed by a processor, causes a system to:
obtain an estimated posture of a sonographer;
obtain, based on data from a sensor, an estimated level of repetitive movements by the sonographer;
obtain an estimated level of pressure applied by the sonographer to a transducer; and
determine a level of cumulative physical strain on the sonographer based on the estimated posture, estimated level of repetitive movements, and estimated level of pressure applied by the sonographer.

10. The tangible non-transitory computer-readable storage medium of claim 9, wherein, when executed by the processor, the computer program further causes the system to:
obtain log-file data from an ultrasound cart; and
estimate the estimated posture of the sonographer based on the log-file data.

11. The tangible non-transitory computer-readable storage medium of claim 10, wherein the log-file data comprises at least one sequence of buttons on the ultrasound cart pushed by the sonographer.

12. The tangible non-transitory computer-readable storage medium of claim 9, wherein, when executed by the processor, the computer program further causes the system to:
determine whether to propose one or more remediation measures to relieve the cumulative physical strain on the sonographer; and
determine one or more feasible remediation measures based on determining to propose one or more remediation measures to relieve the cumulative physical strain.

13. The tangible non-transitory computer-readable storage medium of claim 9, wherein, when executed by the processor, the computer program further causes the system to:
determine a displacement of the sensor, wherein the sensor is attached to an ultrasound probe directly or integrated in or on a cord of the ultrasound probe; and
estimate the estimated level of repetitive movements by the sonographer based on the determined displacement of the sensor.

14. The tangible non-transitory computer-readable storage medium of claim 9, wherein, when executed by the processor, the computer program further causes the system to:
determining a type of each of one or more procedures performed by the sonographer;
determining a setup of an ultrasound system used by the sonographer; and
determining the level of cumulative physical strain on the sonographer based further on the type of the each of the one or more procedures performed by the sonographer and the setup of the ultrasound system used by the sonographer.

15. The tangible non-transitory computer-readable storage medium of claim 9, wherein, when executed by the processor, the computer program further causes the system to:
obtain ultrasound images taken by an ultrasound system;
apply elastography to the ultrasound images taken by the ultrasound system; and
estimate the estimated level of pressure applied by the sonographer to the transducer using elastography based on the elastography applied to the ultrasound images.

16. A system comprising:
a memory that stores instructions; and
a processor that executes the instructions, wherein, when executed by the processor, the instructions cause the system to:
obtain an estimated posture of a sonographer;
obtain, based on data from a sensor, an estimated level of repetitive movements by the sonographer;
obtain an estimated level of pressure applied by the sonographer to a transducer; and
determine a level of cumulative physical strain on the sonographer based on the estimated posture, estimated level of repetitive movements, and estimated level of pressure applied by the sonographer.

17. The system of claim 16, wherein the system is a cloud-based system remote from the sonographer.
